(19) United States
(12) Patent Application Publication
(10) Pub. No.: US 2017/0007130 A1
(43) Pub. Date: Jan. 12, 2017

(54) OPTICAL SENSOR WITH NARROW ANGULAR RESPONSE

(71) Applicant: LFOUNDRY S.r.I., Avezzano (IT)

(72) Inventors: Fabio Spaziani, Avezzano (IT); Andrea Del Monte, Avezzano (IT); Giovanni Margutti, Avezzano (IT); Giovanni De Amicis, Avezzano (IT)

(21) Appl. No.: 14/820,609

(22) Filed: Aug. 7, 2015

(30) Foreign Application Priority Data
Jul. 7, 2015 (IT) 102015000031525

(51) Int. Cl. A61B 5/00; H01L 27/146

(52) U.S. Cl. CPC A61B 5/0059; H01L 27/14643; H01L 27/14603; A61B 5/6801; A61B 2562/0238; A61B 2562/185

(57) ABSTRACT

The present invention relates to an optical sensor that is formed in an integrated circuit based on CMOS technology and that comprises: one or more photocells including one or more photodetector active areas and an optical stack formed on the photodetector active area(s); and light shielding means, that are formed in or on the optical stack, and are configured, for each photocell, to define an angular range around a given incident direction with respect to said photocell, block incident light with incidence angle outside the defined angular range, and allow incident light with incidence angle within the defined angular range to propagate through the optical stack down to a respective photodetector active area; whereby the light shielding means limit angular response of the optical sensor.

### CROSS-REFERENCE TO RELATED APPLICATIONS

The present application claims priority under 35 U.S.C. §119 to Italian Patent Application No. 102015000031525, filed Jul. 7, 2015, the entirety of which is incorporated herein by reference.

### FIELD OF THE INVENTION

The present invention relates to the sector of optical sensors and, specifically, to an optical sensor with narrow angular response. In particular, the present invention finds advantageous, but non-limitative, application in measuring devices, such as wearable biometric monitoring devices.

### BACKGROUND OF THE INVENTION

Recent advances in miniaturization and detection accuracy of optical sensors have allowed the use of such devices in many fields.

For example, optical sensors are currently used in wearable devices to monitor, in a non-invasive fashion, biometric parameters, such as heart rate and blood oxygenation. In particular, these wearable devices, such as watches, arm bands and headsets, can include:

a light source, such as a laser or one or more Light Emitting Diodes (LEDs), configured to emit light having wavelength in visible and/or infrared spectrum so as to illuminate a target volume of a user's body;

a detecting module including one or more optical sensors or detectors, for instance based on Complementary Metal-Oxide-Semiconductor (CMOS) or Charge-Coupled Coupled Device (CCD) technology, and designed to detect light from the target volume of the user's body; and

a processing module configured to determine one or more biometric parameters of the user, such as heart rate and/or blood oxygenation, based on output signals from the detecting module.

As for wearable biometric monitoring devices, it is to be noted that, if the optical sensors do not adhere perfectly to the user's skin, ambient light can affect the detection carried out by the optical sensors. In fact, due to the non-perfect contact between the optical sensors and the user's skin, a gap is interposed therebetween, which allows side light to reach the detectors, thereby interfering with the light from the target volume of the user's body and thus affecting, in particular reducing, Signal-to-Noise Ratio (SNR).

### SUMMARY OF THE INVENTION

A general object of the present invention is, thence, that of overcoming the aforesaid technical problem of wearable biometric monitoring devices due to side light interference.

Moreover, a specific object of the present invention is that of providing an optical sensor with narrow angular response such that to minimize side light interference.

These and other objects are achieved by the present invention in that it relates to an optical sensor, as defined in the appended claims.

In particular, the present invention relates to an optical sensor, that is formed in an integrated circuit based on CMOS technology and that comprises:

one or more photocells including

a. one or more photodetector active areas, and
b. an optical stack formed on the photodetector active area(s); and

light shielding means, that are formed in or on the optical stack, and are configured, for each photocell, to

a. define an angular range around a given incident direction with respect to said photocell,
b. block incident light with incidence angle outside the defined angular range, and
c. allow incident light with incidence angle within the defined angular range to propagate through the optical stack down to a respective photodetector active area;

whereby the light shielding means limit angular response of the optical sensor.

Preferably, the optical sensor comprises a matrix of photocells electrically connected in parallel.

Conveniently, the light shielding means include, for each photocell, a corresponding aperture, that:

is formed through said light shielding means above a respective photodetector active area of said photocell;

defines said angular range with respect to said photocell; and

allows incident light with incidence angle within the defined angular range to propagate through the optical stack down to said respective photodetector active area.

More conveniently, the optical sensor comprises a plurality of photocells, each including a respective photodetector active area; wherein, for each photocell, the corresponding aperture formed through the light shielding means has a width and a height designed so as to define the angular range with respect to said photocell; and wherein the widths and heights of all the apertures limit the angular response of the optical sensor.

More and more conveniently, each photodetector active area has a size, wherein the sizes of the photodetector active areas and the relative position of said photodetector active areas with respect to the apertures formed through the light shielding means further limit the angular response of the optical sensor.

Preferably, the light shielding means are made of one or more metal materials, and/or are formed in one or more metal layers and/or in one or more metal interconnects in the optical stack.

Alternatively, the light shielding means are made of an opaque material, preferably a metal or an opaque polymer, more preferably a black photoresist.

Conveniently, the light shielding means are formed on the optical stack.

### BRIEF DESCRIPTION OF THE DRAWINGS

For a better understanding of the present invention, preferred embodiments, which are intended purely by way of example and are not to be construed as limiting, will now be described with reference to the attached drawings (not to scale), where:

FIG. 1 schematically illustrates working principle of light shielding means according to a first preferred embodiment of the present invention;

FIG. 2 shows an example of photocell with integrated light shielding means according to the first preferred embodiment of the present invention;

FIGS. 3-9 show examples of steps for manufacturing the photocell of FIG. 2;

FIG. 10 shows an example of photocell with light shielding means according to a second preferred embodiment of the present invention; and

FIG. 11 shows a comparison between angular responses of, respectively, a conventional optical sensor and an optical sensor according to the present invention.

### DETAILED DESCRIPTION OF THE INVENTION

The following discussion is presented to enable a person skilled in the art to make and use the invention. Various modifications to the embodiments will be readily apparent to those skilled in the art, without departing from the scope of the present invention as claimed. Thus, the present invention is not intended to be limited to the embodiments shown and described, but is to be accorded the widest scope consistent with the principles and features disclosed herein and defined in the appended claims.

The present invention stems from Applicant's idea of integrating, into an optical sensor based on CMOS technology, light shielding means designed to define an angular range around a given incident direction and to suppress incident light with incidence angle outside the defined angular range. Preferably, said optical sensor equipped with the light shielding means can be integrated into a System-on-a-chip (SoC) along with read-out circuitry, Analogue Signal Processor (ASP), Digital Signal Processor (DSP), Analogue-to-Digital Converter (ADC), etc.

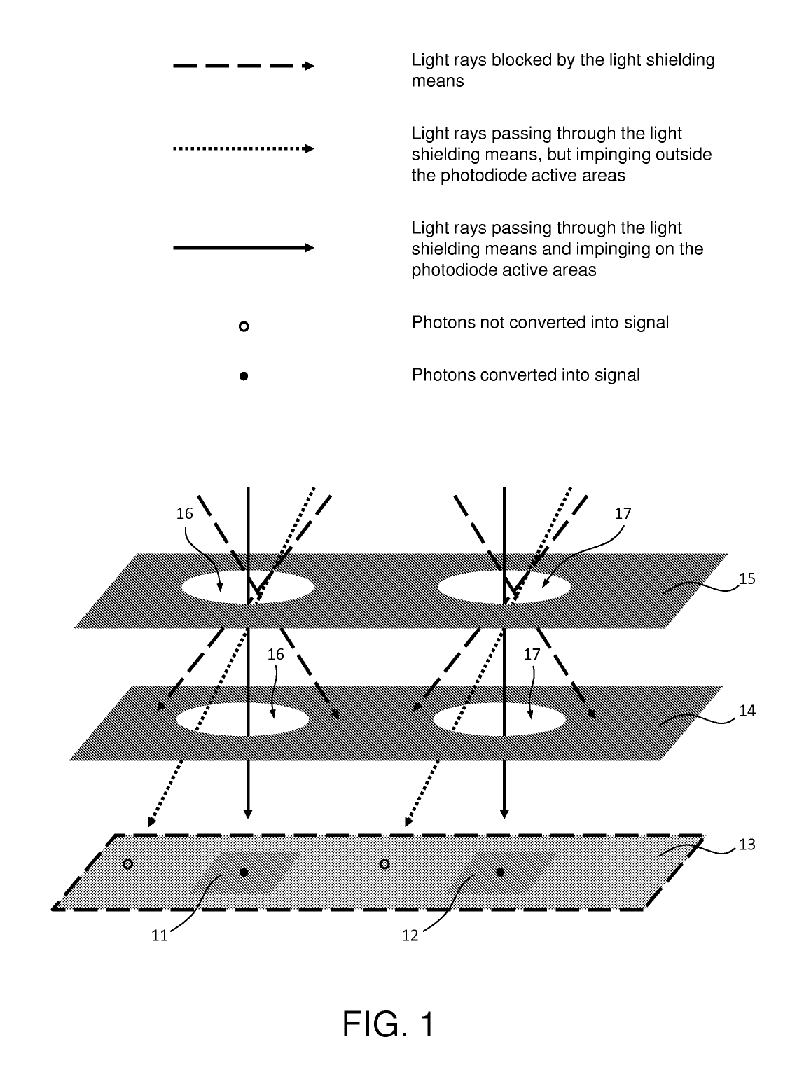

FIG. 1 schematically illustrates working principle of light shielding means according to a first preferred embodiment of the present invention.

In particular, optical sensors realized in CMOS technology generally include single photosensitive units or photocells that, in turn, comprise respective photodetector active areas formed on a semiconductor substrate, as shown in FIG. 1 where photodetector active areas 11 and 12 are sketched on a front side surface 13 of a semiconductor substrate (not shown).

Conveniently, the photodetector active areas 11 and 12 can be realized in the form of photodiode, phototransistor, or photoresistor active areas.

For example, CMOS-based photodiode active areas are realized, in the simplest form, as p-n (or n-p) junctions configured so that the n (or p) regions are depleted from charge carriers (such as electron/hole pairs) and, thence, incident photons generate electron/hole pairs collected by the depletion regions of the photodiodes. As is known, also pnp (or npn) junctions can be conveniently used (so-called “pinned photodiodes”).

Therefore, the photons impinging on the photodetector active areas 11 and 12 are converted into charge carriers producing an output electric signal proportional to the intensity of the incident light, while photons impinging on the front side surface 13 of the semiconductor substrate, outside said photodetector active areas 11 and 12, are also converted into charge carriers, but are not collected by the photodetectors and, thence, do not contribute to the output electric signal.

The light shielding means according to said first preferred embodiment of the present invention include several metal layers configured, for each photocell, to:

define an angular range around a given incident direction with respect to said photocell;

block incident light with incidence angle outside the defined angular range; and

allow incident light with incidence angle within the defined angular range to propagate down to a respective photodetector active area.

In this respect, FIG. 1 shows an example in which two metal layers 14 and 15, in the form of plates, are used as light shielding means. In particular, the metal plates 14 and 15 include first coaxial apertures 16 above the photodetector active area 11 and second coaxial apertures 17 above the photodetector active area 12 so that photons with incident angle higher than a threshold defined by the aspect ratio of the series of apertures 16 and 17 are blocked (in particular are partially reflected and partially absorbed by the metal layers 14 and 15), while photons with incident angle lower than said threshold reach the photodetector active areas 11 and 12 (except for those photons impinging on the front side surface 13 of the semiconductor substrate outside said photodetector active areas 11 and 12).

The apertures 16 and 17 can be circular, squared, rectangular or polygonal.

Conveniently, an antireflective coating can be deposited on the metallic layers 14 and 15 to minimize the reflection and maximize light absorption, thereby avoiding multiple reflections that can cause spurious rays directed toward the photodetector active areas 11 and 12.

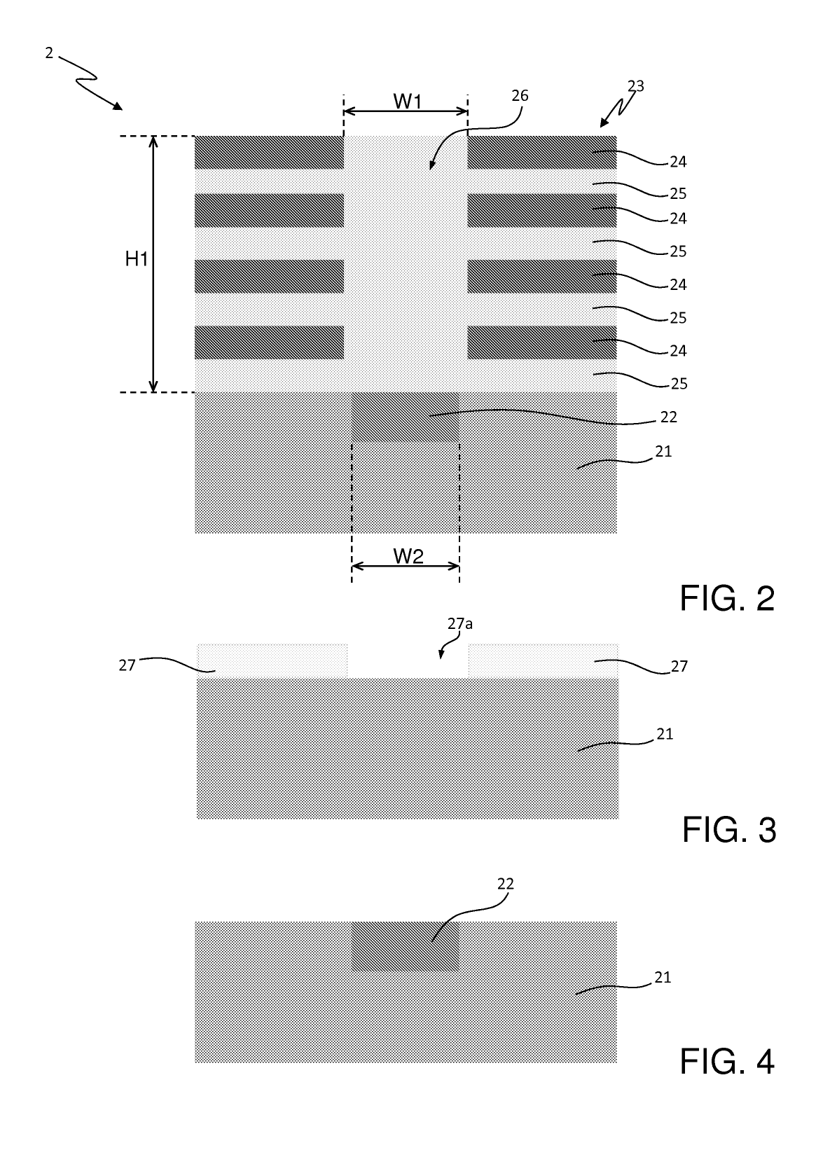

For a better understanding of the present invention, FIG. 2 shows an example of photocell (denoted as a whole by 2) with integrated light shielding means according to said first preferred embodiment. In particular, FIG. 2 is a cross-sectional view of the photocell 2, which includes:

a semiconductor substrate 21 comprising a photodetector active area 22;

an optical stack 23, formed on the semiconductor substrate 21 and comprising four metal layers 24 separated by four inter-level dielectric (ILD) insulating layers 25; and

an aperture 26 formed through the metal layers 24 above the photodetector active area 22.

FIGS. 3-9 show examples of steps for manufacturing the photocell 2.

In particular, FIG. 3 shows a photoresist mask 27 formed on the semiconductor substrate 21 and patterned by a photoligraphic process to expose (through a window 27a) a portion of said semiconductor substrate 21 to one or more subsequent ion implantation processes intended to realize the photodetector active area 22.

FIG. 4 shows the semiconductor substrate 21 on which the photodetector active area 22 has been realized.

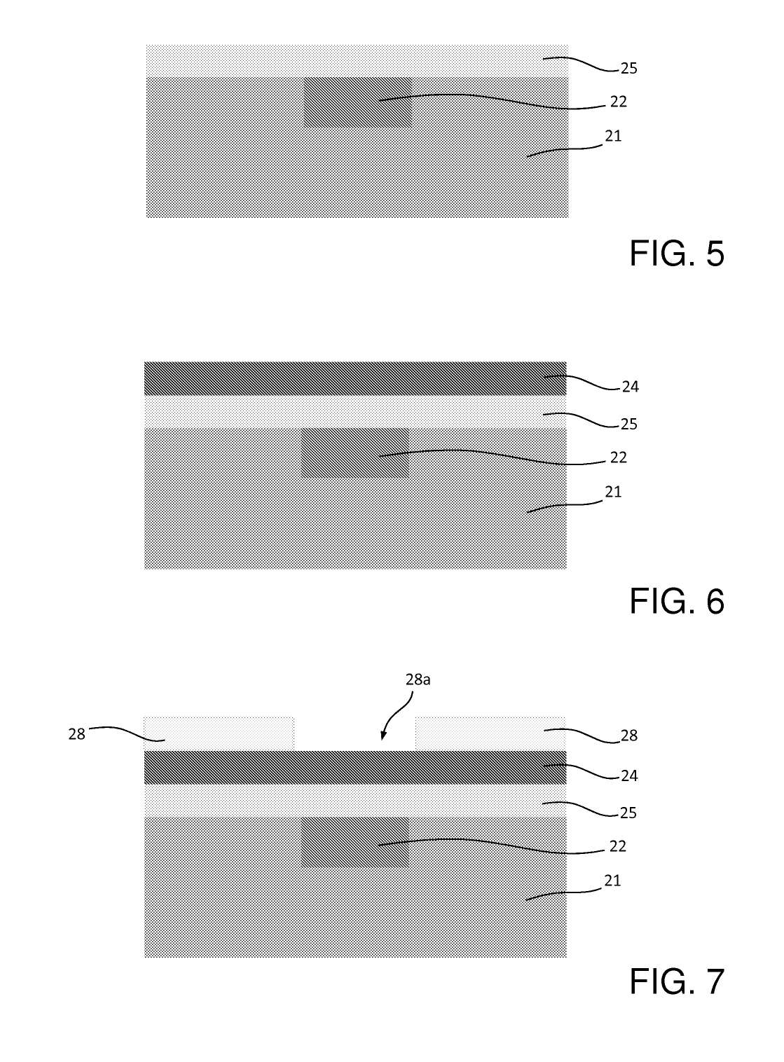

FIG. 5 shows a first ILD insulating layer 25 made from Tetraethyl orthosilicate (commonly known as TEOS) and/or Borophosphosilicate glass (commonly known as BPSG) and deposited (for instance by Chemical Vapor Deposition (CVD)) on the semiconductor substrate 21.

FIG. 6 shows a first metal layer 24 (for example, made up of a stack of three metal materials, such as Ti, TiN and AlCu) deposited (for instance by CVD or Physical Vapor Deposition (PVD)) on the first ILD insulating layer 25.

FIG. 7 shows a photoresist mask 28 patterned by a photoligraphic process to expose (through a window 28a) a portion of the first metal layer 24 (which is above the photodetector active area 22) to a subsequent etch process intended to remove said exposed portion in order to realize the aperture 26.

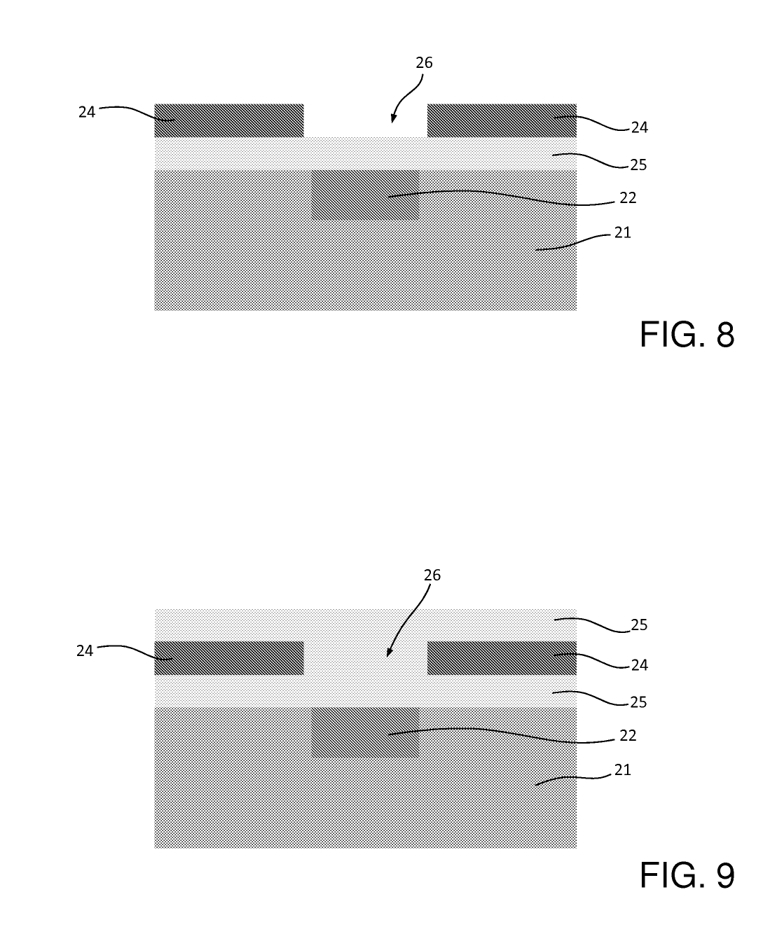

FIG. 8 shows the first metal layer 24 patterned after the etch process so as to have the aperture 26 above the photodetector active area 22. For example, a dry etching stopping at the first ILD insulating layer 25 below said first metal layer 24 can be conveniently used.

FIG. 9 shows a second ILD insulating layer 25 deposited (for instance a SiO₂ film deposited by low-pressure CVD (LPCVD)) on the first metal layer 24 and filling the aperture 26. In detail, FIG. 9 shows the second ILD insulating layer 25 after a planarization process based on Chemical Mechanical Polishing (CMP).

Thence, the optical stack 23 can be manufactured by repeating the above manufacturing steps (in particular, from the step shown in FIG. 6 up to the step shown in FIG. 9).

The above manufacturing process is intended to be used with metal layers based on an aluminum-copper (AlCu) alloy. Otherwise, if metal layers based on copper are used, a manufacturing process based on damascene scheme is conveniently used, wherein a groove pattern is created on the ILD insulating layers 25 and then filled with copper by an electroplating process, followed by a CMP process.

The angular response of the photocell 2 can be tuned by properly adjusting the size (in particular, width and height) of the aperture 26, the size of the photodetector active area 22, and the relative position of the latter with respect to the aperture 26 (for example, as shown in FIG. 2, the width W1 and the height H1 of the aperture 26 and the width W2 of the photodetector active area 22).

An optical sensor can include a single photocell like the one shown in FIG. 2, or, preferably, a matrix of photocells. In this case, the structure shown in FIG. 2 is replicated for each single photocell in the matrix. In particular, in order to maximize the light shielding effect, the height H1 of the optical stack 23 is conveniently at least comparable to the aperture size (e.g., the width W1 of the aperture 26). Thence, preferably, the optical sensor includes a matrix of photocells electrically connected in parallel, thereby simulating the operation of a single big device by means of a plurality of smaller devices which satisfy the above aspect ratio conditions. In this way, the signal output of the optical sensor is given by the sum of the signal outputs of all the photocells.

In summary, according to the first embodiment of the present invention, the light shielding means include metal layers that are integrated in the optical stack of one or more photocells and that can be conveniently shaped as metal routes and/or metal plates or a combination of them.

It is worth noting that, as is known, an image sensor includes an array of pixels and require to address every single pixel of the array for the operations of integration, reset and read-out. Therefore, at least two metal interconnect routes are necessary for this task.

On the contrary, with the above optical sensor including small photocells in parallel that operate, as a whole, as a bigger single device, there is no need to address rows and columns of the photocell matrix. Therefore, one of the metal layers (for example, in the form of a metal plate) acting as light shielding means can be used to address all the photocells, thereby avoiding the need for metal interconnect routes. This results in time and cost optimization in the manufacturing of the optical sensor.

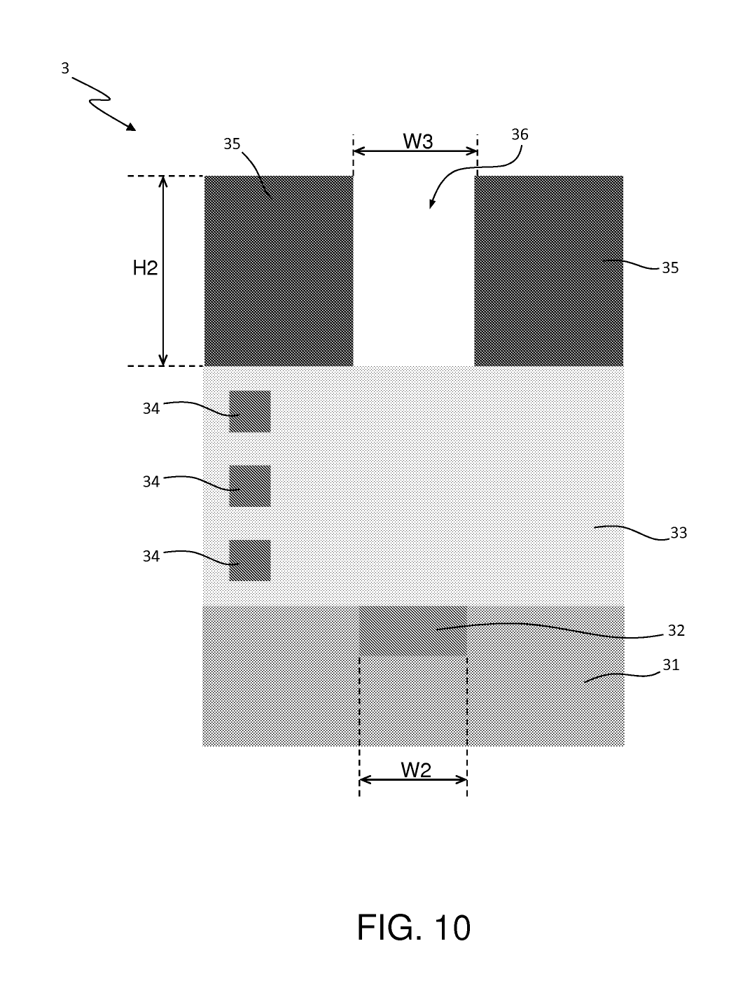

FIG. 10 shows an example of photocell (denoted as a whole by 3) with light shielding means according to a second preferred embodiment of the present invention. In particular, FIG. 10 is a cross-sectional view of the photocell 3, which includes:

a semiconductor substrate 31 comprising a photodetector active area 32;

an optical stack 33, formed on the semiconductor substrate 31 and comprising several transparent dielectric layers (transparent to the wavelength of the radiation to be detected) and, at different levels, metal interconnect routes 34;

an opaque material 35 (opaque to the wavelength of the radiation to be detected), such as an opaque polymer (for example a black photoresist) or a metal, placed on the optical stack 33; and

an aperture 36 through the opaque material 35, that is above the photodetector active area 32 and extends from the top surface of said opaque material 35 to the upper surface of the optical stack 33.

The light shielding effect can be tuned by properly adjusting the height H2 and the width W3 of the aperture 36 in the opaque material 35, the width W2 of the photodetector active area 32, and the relative position of the aperture 36 with respect to the photodetector active area 32.

The advantages of the present invention are clear from the foregoing. In particular, it is important to underline the fact that the light shielding structure according to the present invention allows to limit, in particular to narrow, the angular response of an optical sensor, thereby minimizing the technical problem of wearable biometric monitoring devices due to side light interference.

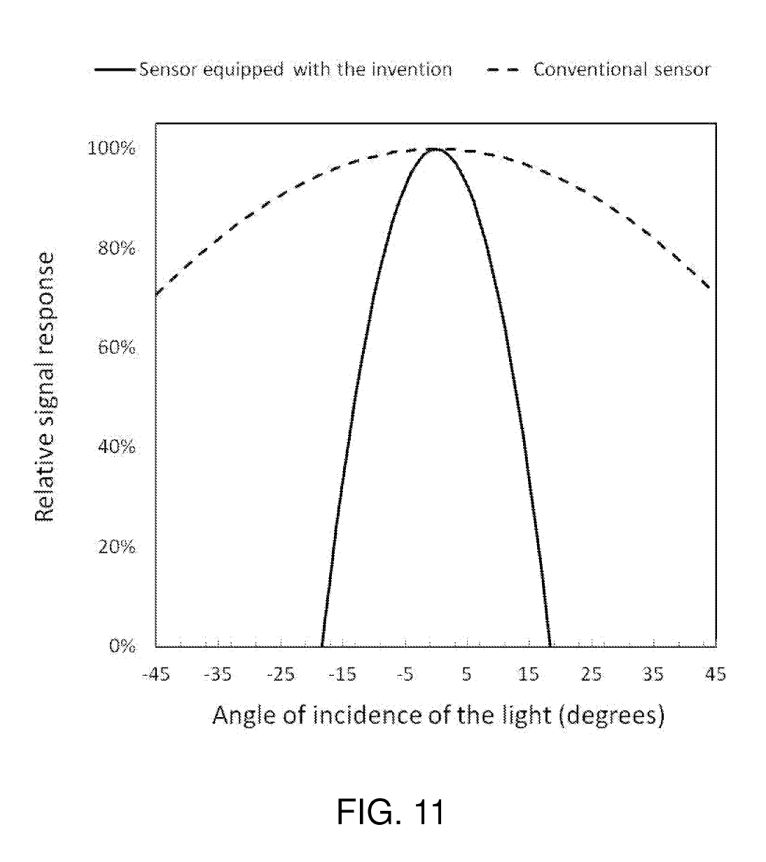

In this respect, FIG. 11 shows a comparison between relative signal responses (with respect to angle of incidence of the light) of a conventional optical sensor and an optical sensor according to the present invention, respectively. In particular, in FIG. 11 each relative signal response represents the signal output normalized to its maximum value. As it is clearly shown in FIG. 11, the optical sensor according to the present invention is characterized by a narrower angular signal response.

Finally, it is clear that numerous modifications and variants can be made to the present invention, all falling within the scope of the invention, as defined in the appended claims. For example, a combination of the two embodiments previously described and illustrated in FIGS. 2 and 10 can be also envisaged.

What is claimed is:

1. An optical sensor, that is formed in an integrated circuit based on CMOS technology and that comprises:
one or more photocells including
one or more photodetector active areas, and
an optical stack formed on the photodetector active area(s); and
light shielding means, that are formed in or on the optical stack, and are configured, for each photocell, to
define an angular range around a given incident direction with respect to said photocell,
block incident light with incidence angle outside the defined angular range, and
allow incident light with incidence angle within the defined angular range to propagate through the optical stack down to a respective photodetector active area;
whereby the light shielding means limit angular response of the optical sensor.

2. The optical sensor of claim 1, comprising a matrix of photocells electrically connected in parallel.

3. The optical sensor according to claim 1, wherein the light shielding means include, for each photocell, a corresponding aperture, that:
is formed through said light shielding means above a respective photodetector active area of said photocell;
defines said angular range with respect to said photocell; and
allows incident light with incidence angle within the defined angular range to propagate through the optical stack down to said respective photodetector active area.

4. The optical sensor of claim 3, comprising a plurality of photocells, each including a respective photodetector active area; wherein, for each photocell, the corresponding aperture formed through the light shielding means has a width and a height designed so as to define the angular range with respect to said photocell; and wherein the widths and heights of all the apertures limit the angular response of the optical sensor.

5. The optical sensor of claim 4, wherein each photodetector active area has a size; and wherein the sizes of the photodetector active areas and the relative position of said photodetector active areas with respect to the apertures formed through the light shielding means further limit the angular response of the optical sensor.

6. The optical sensor according to claim 1, wherein the light shielding means are made of one or more metal materials.

7. The optical sensor according to claim 1, wherein the light shielding means are formed in one or more metal layers in the optical stack.

8. The optical sensor according to claim 1, wherein the light shielding means are formed in one or more metal interconnects in the optical stack.

9. The optical sensor according to claim 1, wherein the light shielding means are made of an opaque material.

10. The optical sensor of claim 9, wherein the opaque material is an opaque polymer or a metal.

11. The optical sensor of claim 10, wherein the opaque polymer is a black photoresist.

12. The optical sensor according to claim 9, wherein the light shielding means are formed on the optical stack.

13. A device for monitoring one or more biometric parameters, that includes an optical sensor as claimed in claim 1.
